(19) United States
(12) Patent Application Publication
(10) Pub. No.: US 2026/0061114 A1
(43) Pub. Date: Mar. 5, 2026

(54) Medical Tray System with Recessed Syringe Slots and Mounting Assembly

(71) Applicants: Tyrone Girdharry, Lancaster, PA (US); Nitin Goyal, Lititz, PA (US); Julius Heyman, Manheim, PA (US)

(72) Inventors: Tyrone Girdharry, Lancaster, PA (US); Nitin Goyal, Lititz, PA (US); Julius Heyman, Manheim, PA (US)

(21) Appl. No.: 19/309,703

(22) Filed: Aug. 26, 2025

Related U.S. Application Data
(60) Provisional application No. 63/688,514, filed on Aug. 29, 2024.

(51) Int. Cl. A61M 5/00

(52) U.S. Cl. CPC A61M 5/008; A61M 2205/14; A61M 2205/3327; A61M 2205/3584; A61M 2207/10; A61M 2209/082

(57) ABSTRACT

An anesthesia, surgical, or medical drug tray system for securely organizing and accessing anesthesia or medical syringes during clinical procedures is disclosed. The system includes a tray body having a plurality of elongated recessed slots, each configured to hold a syringe in a stable, longitudinal orientation with raised ridges to prevent rolling. The tray body is removably seated within a tray pan and mounted via a flexible gooseneck support arm to a clamp attachable to a vertical support structure, such as an IV pole. The gooseneck support arm permits adjustable positioning of the tray for ergonomic access by clinicians. In some embodiments, the system includes an embedded electronic control unit with a micro-controller, weight sensors, and a wireless communication module to detect and transmit syringe usage data. The system supports sterile handling, efficient workflow, and optional real-time inventory monitoring.

### CROSS-REFERENCE TO RELATED APPLICATION

The present application claims priority to, and the benefit of, U.S. Provisional Application No. 63/688,514 which was filed on Aug. 29, 2024, and is incorporated herein by reference in its entirety.

### FIELD OF THE INVENTION

The present invention generally relates to medical device accessories used in surgical, anesthesia, and medical procedures. More specifically, the present invention relates to an anesthesia, surgical, or medical drug tray system configured to securely hold and organize multiple syringes during anesthesia induction and maintenance. The system comprises a rigid or semi-rigid tray body with integrally molded recessed slots, each dimensioned to receive a syringe in a fixed longitudinal orientation. The tray body is removably placed within a tray pan and is supported by a flexible gooseneck support arm connected to a mounting clamp. The clamp is configured to attach to a vertical support such as an IV pole, enabling adjustable positioning of the tray assembly. The tray system is designed or dimensioned to reduce syringe contamination, prevent handling of multiple syringes, and improve workflow efficiency and drug visibility for anesthesia providers during surgical procedures. Accordingly, the present disclosure makes specific reference thereto. Nonetheless, it is to be appreciated that aspects of the present invention are also equally applicable to other like applications, devices, and methods of manufacture.

### BACKGROUND

By way of background, in surgical and clinical settings, anesthesia providers often administer multiple syringes containing various anesthetic drugs. Common practice involves manually holding a plurality of syringes at once, which significantly compromises efficiency, safety, and sterility. In the absence of an organized and secure storage mechanism, anesthesia professionals often place syringes in their pockets, on nearby surfaces, or even on the patient's chest. Such makeshift practices present numerous drawbacks, including the risk of drug mix-ups, contamination, unintentional syringe displacement, and increased cognitive load on the clinician.

Furthermore, current anesthesia workstation configurations do not provide a dedicated, ergonomic solution for the temporary storage of both full and used syringes during induction and maintenance phases of anesthesia. Accordingly, individuals desire a system for storing drug syringes that offers structured syringe organization, easy visibility, single-handed access, and sterile handling during procedures.

Therefore, there exists a long-felt need in the art for a device that provides anesthesia providers with a dedicated, sterile, and hands-free location to organize and access multiple syringes during surgical procedures. There is a long-felt need in the art for a syringe-holding system that reduces reliance on makeshift practices such as placing syringes on a patient's chest or in a clinician's pocket. Additionally, there is a need in the art for an anesthesia tray that is adjustable, compact, and compatible with standard hospital equipment such as IV poles or operating room tables. Furthermore, there is a need for a syringe management system that facilitates both the loading of filled syringes and the return of used syringes in a manner that maintains visibility and minimizes manual handling. Finally, there exists a need for an anesthesia support solution that improves workflow, enhances sterility, and contributes to safer and more efficient anesthesia delivery.

The subject matter disclosed and claimed herein, in one embodiment, comprises an anesthesia, surgical, or medical drug tray system designed to securely hold multiple syringes in an organized and accessible configuration. The system includes a rigid tray body having a generally rectangular (i.e., rectilinear) cuboidal form, with a plurality of longitudinal recessed slots molded into the top surface thereof. Each slot is dimensioned to accommodate a standard anesthesia syringe and includes a proximal end for the syringe plunger and a narrower distal end for the syringe barrel. The slots further incorporate raised ridges or tabs to prevent syringe rolling and provide light retention. The tray body is removably placed into a tray pan, which can also serve as a spill-resistant basin or sterile containment area. The tray pan is supported by a flexible gooseneck support arm affixed to a mounting clamp, enabling the entire assembly to be positioned as needed on standard IV poles or table rails.

In one embodiment, the anesthesia, surgical, or medical drug tray system utilizes a stainless-steel gooseneck support arm that provides both rigid support and flexible positioning. The arm is connected to a mounting clamp configured with internal gripping ridges, enabling a non-slip attachment to cylindrical vertical supports. The clamp may be manually tightened or adjusted along the height of the pole, enabling optimal ergonomic access by the anesthesia provider. The tray body itself is removably seated within the tray pan for case of cleaning, sterilization, or replacement, and may be disposed of entirely in single-use scenarios.

In this manner, the anesthesia, surgical, or medical drug tray system of the present invention addresses longstanding deficiencies in clinical syringe handling by providing a stable, hygienic, and adjustable solution. The system reduces the need for anesthesia providers to hold multiple syringes simultaneously or rely on unsafe placement surfaces. The system enhances procedural workflow by enabling planned syringe sequencing, improves visual access to drug labels and doses, and minimizes contamination risks. The system is compatible with existing hospital infrastructure and can be adapted for reusable or disposable use cases.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an anesthesia, surgical, or medical drug tray system. The system comprises a tray body having a generally rectangular (i.e., rectilinear) cuboidal configuration formed from an autoclavable plastic material. The tray body includes a plurality of elongated recessed slots that extend longitudinally along a top surface thereof. Each of the recessed slots includes a proximal end configured to receive a plunger flange of a syringe, a distal end configured to receive a syringe barrel/portion, and one or more raised ridges configured to inhibit the syringe from rolling or sliding. The system further includes a tray pan configured to receive and support the tray body, wherein the tray pan has a geometric shape corresponding to the tray body to permit flush placement of the tray body therein. A flexible gooseneck support arm is connected at a first end to a side surface of the tray pan, and a second end of the gooseneck support arm is connected to a mounting clamp. The mounting clamp is configured to removably attach the anesthesia, surgical, or medical drug tray system to a vertical support structure. The gooseneck support arm is further configured to enable positional adjustment of the tray pan in three-dimensional space while providing rigid support to maintain stability during clinical use.

In another embodiment, a system for supporting anesthesia drug administration in a clinical environment is provided. The system comprises a tray assembly including a tray body having a plurality of elongated recessed slots, each recessed slot being sized to accommodate a medical syringe in a fixed longitudinal orientation such that the syringe plunger is positioned at a proximal end and the syringe barrel is positioned at a distal end of the slot. The tray body is removably supported by a tray pan. The tray assembly further comprises a gooseneck support arm extending from the tray pan to a mounting clamp. The mounting clamp is configured to be securely fastened to a vertically extending support structure, such as an intravenous (IV) pole. The gooseneck support arm is configured to enable adjustment of the position of the tray assembly relative to a clinician, thereby enabling single-handed retrieval of syringes from the tray during a surgical or medical procedure.

In one embodiment, a method of organizing and using drug syringes in a surgical environment is disclosed. The method comprises the step of affixing a tray assembly to a vertical support using a mounting clamp and a flexible gooseneck support arm, wherein the tray assembly includes a tray body removably seated within a tray pan. The method further includes placing a plurality of pre-filled, pre-labeled drug syringes into recessed slots formed on the tray body, each slot being configured to hold a single syringe in a longitudinal orientation. The tray assembly is then adjusted to a desired ergonomic working position for a clinician using the gooseneck support arm. During a surgical or anesthesia procedure, the clinician retrieves one or more syringes from the tray body in a single-handed manner. After the completion of the procedure, the tray body is removed from the tray assembly and either sterilized or disposed of based on clinical protocols.

In yet another embodiment, an anesthesia, surgical, or medical drug tray system with electronic monitoring and wireless communication capability is disclosed. The system comprises a tray body having a plurality of elongated recessed slots, each slot being configured to receive and securely hold a syringe. The system includes a micro-controller operably coupled to the tray body and configured to detect placement or removal events associated with the syringes. A weight sensor is provided in communication with each recessed slot and is configured to detect changes in mass indicative of whether a syringe is present, absent, or partially filled. The system also includes a wireless communication module operably connected to the micro-controller. The wireless communication module is configured to transmit usage data such as syringe presence, timestamps, or activity logs to a remote system. Additionally, the tray system includes a mounting mechanism comprising a gooseneck support arm and a clamp, the clamp being configured to attach the tray system to a vertical support structure.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of anesthesia, surgical, or medical drug tray system of the present invention in accordance with the disclosed structure;

FIG. 2 illustrates a partial perspective view showing the tray body detached from the tray pan in accordance with the disclosed structure;

FIG. 3 illustrates a perspective view showing the anesthesia, surgical, or medical drug tray assembly affixed to a vertical support structure in accordance with one embodiment of the present invention;

FIG. 4 illustrates a top perspective view of the anesthesia, surgical, or medical drug tray in use, with various syringes placed in designated recessed slots in accordance with the disclosed structure;

FIG. 5 illustrates a perspective view showing a medical professional accessing a syringe from the tray body while the tray assembly is mounted on an IV pole during a clinical procedure in accordance with one embodiment of the present invention;

FIG. 6 illustrates a flow chart depicting the process of use of the anesthesia, surgical, or medical drug tray assembly of the present invention in accordance with the disclosed structure; and

FIG. 7 illustrates a block diagram of an embedded electronic control unit (ECU) integrated into the anesthesia, surgical, or medical drug tray system for smart monitoring, tracking, and communication functionalities in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that provides anesthesia providers with a dedicated, sterile, and hands-free location to organize and access multiple syringes during surgical procedures. There is a long-felt need in the art for a syringe-holding system that reduces reliance on makeshift practices such as placing syringes on a patient's chest or in a clinician's pocket. Additionally, there is a need in the art for an anesthesia tray that is adjustable, compact, and compatible with standard hospital equipment such as IV poles or operating room tables. Furthermore, there is a need for a syringe management system that facilitates both the loading of filled syringes and the return of used syringes in a manner that maintains visibility and minimizes manual handling. Finally, there exists a need for an anesthesia support solution that improves workflow, enhances sterility, and contributes to safer and more efficient anesthesia delivery.

The present invention, in one exemplary embodiment, is an anesthesia, surgical, or medical drug tray system with electronic monitoring and wireless communication capability. The system comprises a tray body having a plurality of elongated recessed slots, each slot being configured to receive and securely hold a syringe. The system includes a micro-controller operably coupled to the tray body and configured to detect placement or removal events associated with the syringes. A weight sensor is provided in communication with each recessed slot and is configured to detect changes in mass indicative of whether a syringe is present, absent, or partially filled. The system also includes a wireless communication module operably connected to the micro-controller. The wireless communication module is configured to transmit usage data such as syringe presence, timestamps, or activity logs to a remote system. Additionally, the tray system includes a mounting mechanism comprising a gooseneck support arm and a clamp, the clamp being configured to attach the tray system to a vertical support structure.

Reference will now be made in detail to the present preferred embodiments of the invention, examples of which are illustrated in the accompanying drawings. Wherever possible, the same reference numerals are used in the drawings and the description to refer to the same or like parts.

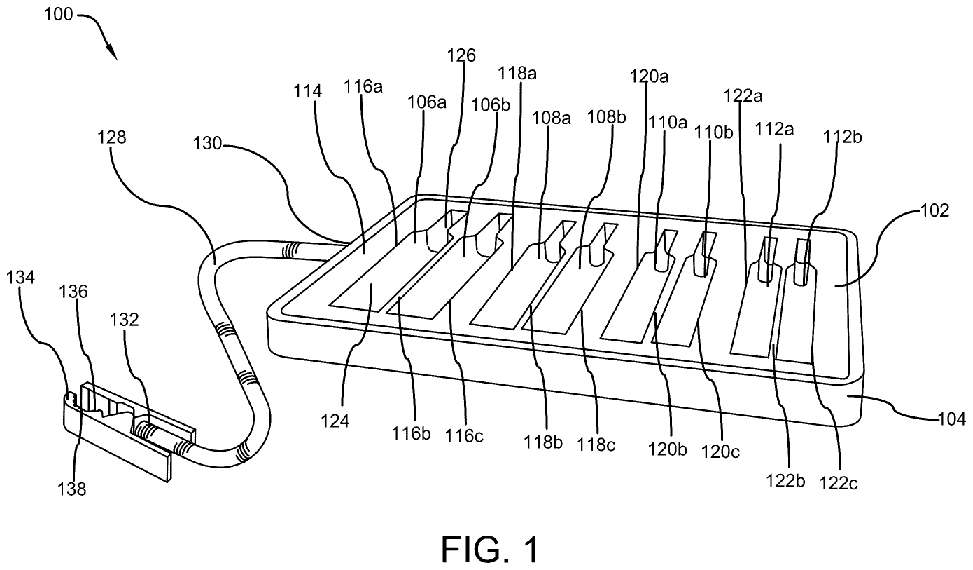

Referring initially to the drawings, FIG. 1 illustrates a perspective view of anesthesia, surgical, or medical drug tray system of the present invention in accordance with the disclosed structure. The anesthesia, surgical, or medical drug tray system 100 of the present invention is designed or dimensioned for securely handling anesthesia drugs in a surgical environment for improved safety and efficiency in operating rooms. The anesthesia, surgical, or medical drug tray system 100 stores the syringes during anesthesia induction and eliminates the need to place syringes on the patient or hold them by hand. More specifically, the anesthesia, surgical, or medical drug tray assembly/system 100 includes a tray body 102 preferably formed from a medical-grade, autoclavable plastic, such as polypropylene (PP) or polycarbonate (PC). The tray body 102 is removably placed in a tray pan 104. The tray pan 104 is generally rectangular (i.e., rectilinear) or square but can be made in any geometric shape.

The tray body 102 has generally rectangular (i.e., rectilinear) cuboidal configuration for storage in the tray pan 104 and for safety during use of the tray body 102. A plurality of elongated recessed indentations or slots 106a-b, 108a-b, 110a-b, 112a-b extend longitudinally along the top surface 114 of the tray body 102. Each slot of the plurality of slots is dimensioned to accommodate a standard anesthesia drug syringe. Further, each slot includes raised ridges for preventing syringes from sliding or rolling. As illustrated, the slots 106a,b have the raised ridges 116a-c, the slots 108a-b have the raised ridges 118a-c, the slots 110a-b have the raised ridges 120a-c, and the slots 112a-b have the raised ridges 122a-c. The recessed slots 106a-112b are integrally molded into the tray body 102 and are shaped to enable easy insertion and removal with light resistance to keep the syringes stable.

Each slot includes a proximal end 124 for storing hub or plunger flange of a syringe and a narrow distal end 126 for storing the syringe portion/barrel of an anesthesia syringe (shown as an example only for the slot 106a). The slots can store a variety of syringes, differing in lengths, needle gauges, and volume. The tray body 102 also provides clear visibility of syringe contents and labels, thereby helping in drug identification during surgery.

The anesthesia, surgical, or medical drug tray system 100 includes gooseneck support arm 128, preferably made from flexible stainless-steel coil. The gooseneck support arm 128 is affixed to a side surface 130 of the tray pan 104 and a free end 132 of the gooseneck support arm 128 is attached to a mounting clamp 134. The mounting clamp 134 can be used to attach the anesthesia, surgical, or medical drug tray system 100 to a vertical support such as an IV pole. The gooseneck support arm 128 enables positional adjustment of the tray pan 104 in different directions (i.e., X, Y, and Z-axis movement) while manually overcoming the frictional force of the gooseneck support arm 128. The gooseneck support arm 128, when not manually manipulated, rigidly fixes a desired position of the tray pan 104.

The mounting clamp 134 is made from high-strength polymer or anodized aluminum and is designed or dimensioned to fit snugly around a vertical cylindrical post. The mounting clamp 134 includes a plurality of gripping ridges 136 along the internal profile 138 thereof to increase friction and reduce slipping.

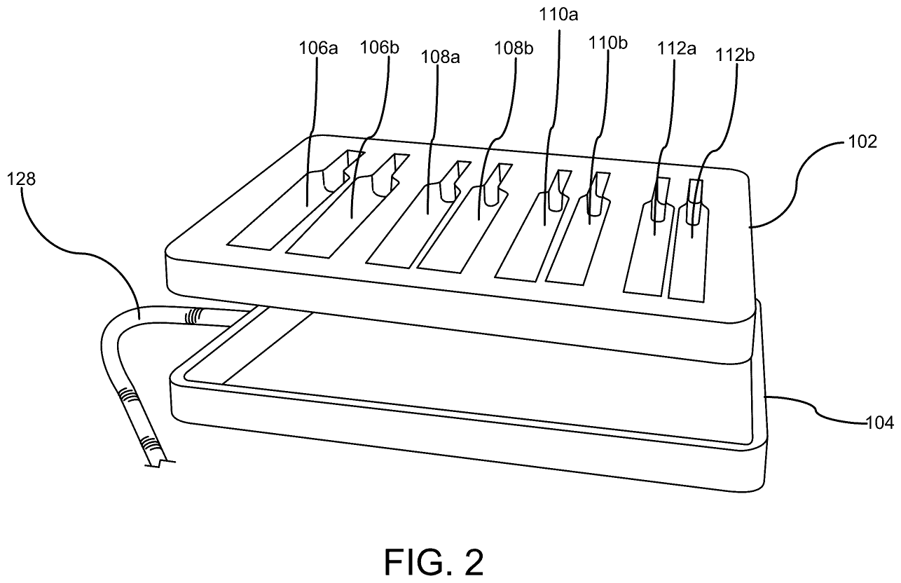

FIG. 2 illustrates a partial perspective view showing the tray body detached from the tray pan in accordance with the disclosed structure. The tray body 102 forms the primary surface for syringe placement in the recessed slots 106a-112b. The tray body 102 is sterilizable and is dimensionally identical to the tray pan 104 for flushing into the tray pan 104. The tray pan 104 can also serve to catch drips or residue from syringes and act as sterile storage compartment. The removable tray body 102 provides easy loading and unloading of syringes and can be replaced based on the requirements of a user. The tray body 102 can be replaced or cleaned separately and can also be disposed as needed.

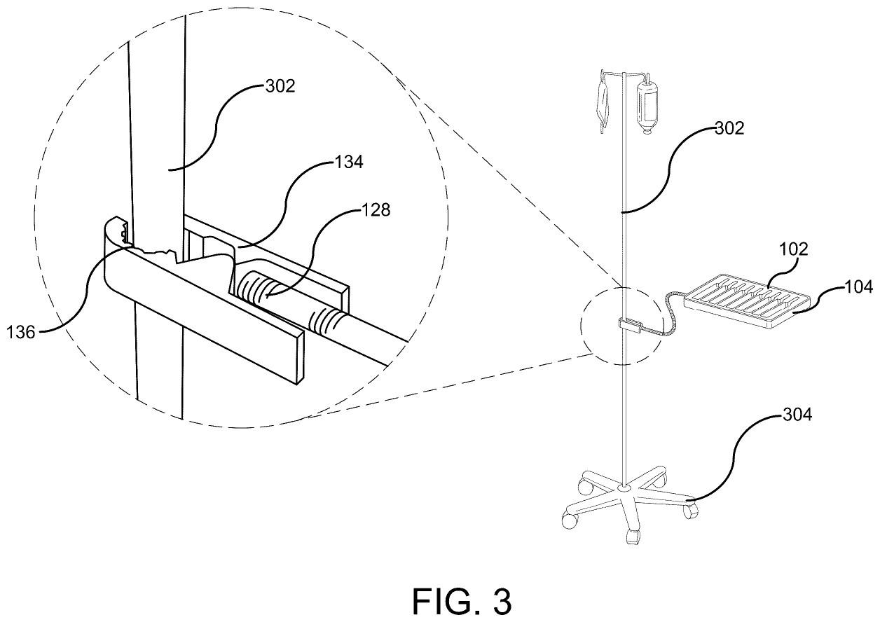

FIG. 3 illustrates a perspective view showing the anesthesia, surgical, or medical drug tray assembly affixed to a vertical support structure in accordance with one embodiment of the present invention. The anesthesia, surgical, or medical drug tray assembly 100 can be affixed to a vertical pole 302 such as an IV pole (having a base 304) using the mounting clamp 134. The gripping ridges 136 of the mounting clamp 134 apply lateral force to the pole 302 for providing a non-slip, secure fit of the mounting clamp 134. Further, the mounting clamp 134 can be adjusted along the length of the pole 302.

The gooseneck support arm 128 and mounting clamp 134 enables the tray pan 104 and the tray body 102 to be kept in various positions and surfaces enabling easy accessibility of the stored drug syringes. The anesthesia, surgical, or medical drug tray assembly 100 is mounted in a non-invasive approach and avoids permanent modification to standard hospital equipment.

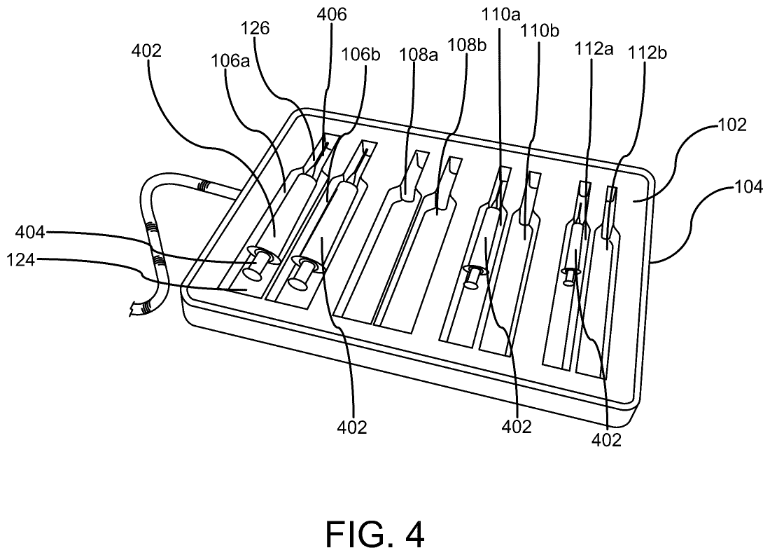

FIG. 4 illustrates a top perspective view of the anesthesia, surgical, or medical drug tray in use, with various syringes placed in designated recessed slots in accordance with the disclosed structure. The recessed slots/indentations 106a-112b can be used to removably store the syringes 402 of varying sizes. Preferably, the recessed slots are equally spaced but can be spaced at different gaps. In the preferred embodiments, all the recessed slots are aligned parallel to each other. The plunger 404 of each syringe 402 is positioned in the proximal end 124 and the syringe portion 406 is positioned in the narrow distal end 126. The transparent syringe barrels, including visible plunger stops and measured dose markers, remain clearly distinguishable while the syringes 402 are stored in the tray body 102, helping rapid drug identification.

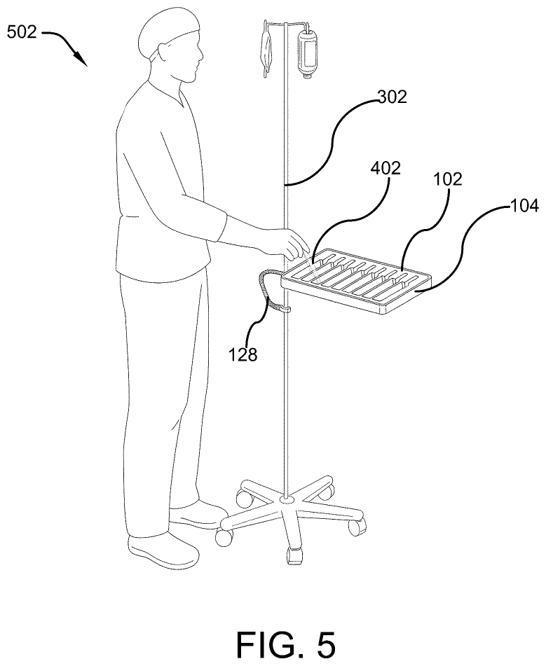

FIG. 5 illustrates a perspective view showing a medical professional accessing a syringe from the tray body while the tray assembly is mounted on an IV pole during a clinical procedure in accordance with one embodiment of the present invention. A clinician 502 in their natural standing position such as during a medical procedure can retrieve a syringe 402 from any of the recessed slots of the tray assembly 100. Based on the preferences of the clinician 502, the tray assembly 100 can be mounted using the mounting clamp 134 at any height along the length of the pole 302. Further, the tray assembly 100 can be placed at any appropriate X, Y, and Z axis position using the gooseneck support arm 128.

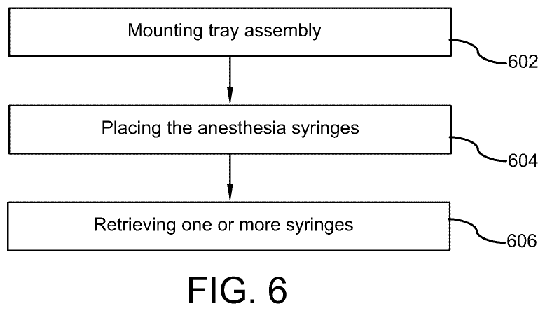

FIG. 6 illustrates a flow chart depicting the process of use of the anesthesia, surgical, or medical drug tray assembly of the present invention in accordance with the disclosed structure. Initially, the tray assembly 100 is mounted securely using the mounting clamp 134 to a pole such as a standard IV pole or operating room table rail (Step 602). Then, the pre-labeled, sterile syringes according to a surgical plan are placed into the individual recesses of the tray body 102 (Step 604). The syringes can be placed in a planned sequence such as induction, maintenance, and rescue for efficient workflow. Thereafter, during anesthesia induction or surgical procedures, a clinician such as an anesthesiologist retrieves syringes single-handedly from the tray assembly (Step 606). The tray assembly 100 remains hands-free, elevated, and does not interfere with patient space. Post procedure, the tray assembly can be detached, and the tray body may be disposed or sterilized.

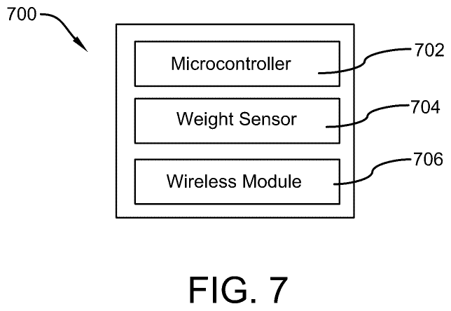

FIG. 7 illustrates a block diagram of an embedded electronic control unit (ECU) integrated into the anesthesia, surgical, or medical drug tray system for smart monitoring, tracking, and communication functionalities in accordance with one embodiment of the present invention. The control unit 700 can be housed within or coupled to the tray assembly 100 and is configured for detecting the presence or removal of syringes and transmitting it wirelessly to a remote monitoring system. A micro-controller 702 is configured to collect and interpret syringe placement or removal events. The micro-controller 702 is a low-power embedded processor suitable for clinical environments.

A weight sensor 704 is configured to detect changes in mass associated with the placement or removal of syringes from specific tray slots. Each slot may have a dedicated load cell or piezoelectric pressure sensor to detect which slot is active and whether the syringe is full, partially filled, or empty.

A wireless communication module 706 enables wireless data communication between the tray assembly and external systems and may use Bluetooth®, Wi-Fi®, Zigbee®, or proprietary RF protocols. The wireless communication module 706 may transmit information such as syringe removal timestamps and slot activity logs.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “anesthesia, surgical, or medical drug tray system”, “anesthesia, surgical, or medical drug tray assembly”, “tray assembly”, and “system” are interchangeable and refer to the anesthesia, surgical, or medical drug tray assembly 100 of the present invention.

Notwithstanding the forgoing, the anesthesia, surgical, or medical drug tray assembly 100 of the present invention can be of any suitable configuration as is known in the art without affecting the overall concept of the invention, provided that it accomplishes the above stated objectives. One of ordinary skill in the art will appreciate that the anesthesia, surgical, or medical drug tray assembly 100 as shown in the FIGS. are for illustrative purposes only, and that many other configurations of the anesthesia, surgical, or medical drug tray assembly 100 are well within the scope of the present disclosure. Although the dimensions of the anesthesia, surgical, or medical drug tray assembly 100 are important design parameters for user convenience, the anesthesia, surgical, or medical drug tray assembly 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical drug tray system for drug syringes comprising:
a tray body;
a tray pan;
a gooseneck support arm;
a mounting clamp;
a plurality of drug syringes; and
a support pole;
wherein said tray body is selectively placeable in said tray pan and selectively detachable from said tray pan;
wherein said tray body comprising a cuboidal configuration for placement in said tray pan;
wherein said tray body comprising a plurality of elongated recessed slots extending longitudinally along a top surface of said tray body;
wherein each recessed slot of said plurality of elongated recessed slots is dimensioned to accommodate a drug syringe;
wherein an adjacent pair of elongated recessed slots comprising at least a first outer raised ridge, a second outer raised ridge, and a third center raised edge between said adjacent pair of elongated recessed slots to prohibit movement of a pair of drug syringes placed in said adjacent pair of elongated recessed slots;
wherein said gooseneck support arm comprising a flexible stainless-steel coil; and
further wherein said gooseneck support arm is affixed to a side surface of said tray pan at one end and is attached to said mounting clamp at another end for attaching said tray pan at a selective height to said support pole.

2. The medical drug tray system for drug syringes of claim 1, wherein said tray body having a material selected from the group consisting of an autoclavable plastic, a polypropylene (PP), and a polycarbonate (PC).

3. The medical drug tray system for drug syringes of claim 2, wherein said plurality of elongated recessed slots are equally spaced and parallel to each other along said top surface of said tray body.

4. The medical drug tray system for drug syringes of claim 3, wherein said plurality of elongated recessed slots are integrally molded into said tray body.

5. The medical drug tray system for drug syringes of claim 4, wherein said tray pan comprising a rectilinear configuration; and further wherein said tray body comprising a rectilinear configuration.

6. The medical drug tray system for drug syringes of claim 4, wherein said each recessed slot comprises a proximal end for storing a plunger flange of said drug syringe and a distal end for storing a barrel portion of said drug syringe, and further wherein a width of said proximal end is greater than a width of said distal end.

7. The medical drug tray system for drug syringes of claim 6, wherein said plurality of drug syringes comprising differing lengths, needle gauges, and volumes.

8. The medical drug tray system for drug syringes of claim 6, wherein said flexible stainless-steel coil of said gooseneck support arm provides X-axis, Y-axis, and Z-axis directional manual adjustment and rigid support of said tray pan.

9. The medical drug tray system for drug syringes of claim 8, wherein said mounting clamp comprising a material selected from the group consisting of a polymer and an anodized aluminum.

10. The medical drug tray system for drug syringes of claim 9, wherein said support pole is selected from the group consisting of an IV pole and an operating room table rail, and further wherein said mounting clamp comprising dimensions to fit snugly around said support pole.

11. The medical drug tray system for drug syringes of claim 10, wherein said mounting clamp comprising a plurality of gripping ridges along an internal profile to increase gripping friction around said IV pole.

12. The medical drug tray system for drug syringes of claim 2, wherein said plurality of elongated recessed slots are unequally spaced and parallel to each other along said top surface of said tray body.

13. The medical drug tray system for drug syringes of claim 11, wherein said tray body is sterilizable.

14. The medical drug tray system for drug syringes of claim 11, wherein said tray body is dimensioned to fit flush into said tray pan.

15. The medical drug tray system for drug syringes of claim 11, wherein said tray body is disposable.

16. A medical drug tray system for drug syringes comprising:
a tray body;
a tray pan;
a gooseneck support arm;
a mounting clamp;
a plurality of drug syringes; and
a support pole;
wherein said tray body is selectively placeable in said tray pan and selectively detachable from said tray pan;
wherein said tray body comprising a cuboidal configuration for placement in said tray pan;
wherein said tray body comprising a plurality of elongated recessed slots extending longitudinally along a top surface of said tray body;
wherein each recessed slot of said plurality of elongated recessed slots is dimensioned to accommodate a drug syringe;
wherein an adjacent pair of elongated recessed slots comprising at least a first outer raised ridge, a second outer raised ridge, and a third center raised edge between said adjacent pair of elongated recessed slots to prohibit movement of a pair of drug syringes placed in said adjacent pair of elongated recessed slots;
wherein said gooseneck support arm comprising a flexible stainless-steel coil;
wherein said gooseneck support arm is affixed to a side surface of said tray pan at one end and is attached to said mounting clamp at another end for attaching said tray pan at a selective height to said support pole;
wherein said each recessed slot comprises a proximal end for storing a plunger flange of said drug syringe and a distal end for storing a barrel portion of said drug syringe;
wherein a width of said proximal end is greater than a width of said distal end;
wherein said plurality of drug syringes comprising differing lengths, needle gauges, and volumes of said plurality of drug syringes; and
further wherein said flexible stainless-steel coil of said gooseneck support arm provides X-axis, Y-axis, and Z-axis directional manual adjustment and rigid support of said tray pan.

17. A medical drug tray system for drug syringes comprising:
a tray body;
a tray pan;
a gooseneck support arm;
a mounting clamp;
a plurality of drug syringes;
a support pole; and
an electronic control unit;
wherein said tray body is selectively placeable in said tray pan and selectively detachable from said tray pan;
wherein said tray body comprising a cuboidal configuration for placement in said tray pan;
wherein said tray body comprising a plurality of elongated recessed slots extending longitudinally along a top surface of said tray body;
wherein each recessed slot of said plurality of elongated recessed slots is dimensioned to accommodate a drug syringe;
wherein an adjacent pair of elongated recessed slots comprising at least a first outer raised ridge, a second outer raised ridge, and a third center raised edge between said adjacent pair of elongated recessed slots to prohibit movement of a pair of drug syringes placed in said adjacent pair of elongated recessed slots;
wherein said gooseneck support arm comprising a flexible stainless-steel coil;
wherein said gooseneck support arm is affixed to a side surface of said tray pan at one end and is attached to said mounting clamp at another end for attaching said tray pan at a selective height to said support pole;
wherein said each recessed slot comprises a proximal end for storing a plunger flange of said drug syringe and a distal end for storing a barrel portion of said drug syringe;
wherein a width of said proximal end is greater than a width of said distal end; and
further wherein said electronic control unit coupled to said tray pan and said tray body for detecting a presence or removal of one or more of said plurality of drug syringes.

18. The medical drug tray system for drug syringes of claim 17, wherein said electronic control unit comprising a weight sensor configured to detect changes in mass associated with the placement or removal of one or more of said plurality of drug syringes from said tray body.

19. The medical drug tray system for drug syringes of claim 18, wherein said weight sensor is a piezoelectric pressure sensor to detect occupancy information of said plurality of elongated recessed slots.

20. The medical drug tray system for drug syringes of claim 19 further comprising a wireless communication module for wireless data communication with said electronic control unit to transmit said occupancy information selected from the group consisting of a drug syringe removal timestamp and activity logs of said plurality of elongated recessed slots.
